(19) United States
(12) Patent Application Publication
(10) Pub. No.: US 2002/0031841 A1
(43) Pub. Date: Mar. 14, 2002

(54) Colorimetric reagent

(76) Inventors: Sanford A. Asher, Pittsburgh, PA (US); Chad E. Reese, Pittsburgh, PA (US)

Correspondence Address: BAKER BOTTS, L.L.P., 30 ROCKEFELLER PLAZA, NEW YORK, NY, 10112, US

(21) Appl. No.: 09/865,348

(22) Filed: May 25, 2001

Related U.S. Application Data
(63) Continuation-in-part of application No. 09/753,592, filed on Jan. 3, 2001.
Application No. 09/753,592 is a continuation of application No. 09/111,610, filed on Jul. 7, 1998, now Pat. No. 6,187,599.
Application No. 09/111,610 is a continuation of application No. 08/819,240, filed on Mar. 17, 1997, now Pat. No. 5,854,078.
Application No. 08/819,240 is a continuation-in-part of application No. 08/743,816, filed on Nov. 6, 1996, now Pat. No. 5,898,004.

(51) Int. Cl. G01N 33/543

(52) U.S. Cl. 436/518

(57) ABSTRACT

A novel colorimetric reagent is disclosed which can be used to sense a wide variety of analytes. The novel colorimetric reagent of the present invention is based in part on sensor devices composed of a crystalline colloidal array (CCA) polymerized in a hydrogel, in that the colorimetric reagent is obtained by preparing fragments from the polymerized crystalline colloidal array (PCCA) which are dispersed, for example, in a medium, such a solvent, or in the atmosphere. The hydrogels are characterized as being capable of shrinking and swelling in response to specific stimuli applied thereto. As the hydrogels shrink or swell, the lattice structure of the CCA embedded therein changes, thereby changing the wavelength of light diffracted by the CCA. When the PCCA fragments are in a dispersion in a medium, the diffraction from the dispersion is used to determine the concentration of analyte. The diffraction of the dispersed fragments results in essentially a powder pattern for the diffraction. The powder pattern diffraction band edge shifts in proportion to analyte concentration.

The colorimetric reagents of the present invention may be specific in that they may be modified to react with only one species or a family of species. These solutions have various applications in areas including, for example, environmental and chemical systems, chemomechanical systems, sensor devices, detection of chemicals used in the environment, detection of chemical or biological weapons, and medical diagnostic tools. Various methods for making and using the colorimetric reagents are also disclosed.

### BACKGROUND OF THE INVENTION

The present invention generally relates to optical, hydrogel-based colorimetric reagents that utilize the diffraction properties of crystalline colloidal arrays. More specifically, the present invention relates to colorimetric reagents comprising polymerized crystalline colloidal arrays (PCCAs) or whose diffraction wavelengths change in response to a variety of specific stimuli. These PCCAs have application as both a colorimetric reagent and a remote sensor device in numerous chemical, environmental, and biomedical technologies.

Charged colloidal particles, when suspended in water, form a stable, crystalline dispersion due to interparticle coulomb repulsion forces. The property of structural ordering in such dispersions has been exploited in making devices such as narrow band optical rejection filters. The Bragg diffraction phenomena in such colloidal suspensions have been useful in spectroscopy and Bragg diffraction techniques. It has been found that mesoscopic, crystalline structures can have many practical applications as optical filters in military, space, medical and research uses. In many such instances, it is necessary or desirable to filter narrow bands of selected wavelengths from a broader spectrum of incident radiation. Crystalline structures, or crystalline colloidal arrays (CCA), and their use in optical filtering devices are disclosed, for example, in U.S. Pat. Nos. 4,627,689 and 4,632,517.

Similar devices, in which a CCA is embedded in a polymer matrix, have also been disclosed. For example, U. S. Pat. Nos. 5,368,781 and 5,266,238 disclose tunable, narrow band radiation filters comprising a crystalline colloidal array of charged particles fixed in a hydrogel film. Methods for filtering incident radiation using these filters are also disclosed.

U.S. Pat. Nos. 5,330,685, 5,338,492 and 5,342,552 discuss narrow band radiation filters comprising a CCA of charged particles in a polymeric hydrogel. U.S. Pat. No. 5,281,370 also discloses a method of making a solid radiation filter material including one embodiment in which the particles in the array are fused together by polymerization.

Various sensor devices are also reported in the art. Schalkhammer, et al., disclose an optical sensor that utilizes the concept of pH-dependent swelling of special polymers. See Schalkhammer, et al. , “The Use of Metal-island-coated pH Sensitive Swelling Polymers for Biosensor Applications”, Sensors and Actuators B, Vol. 24-25, pp. 166-172 (1995). Conductimetric sensor devices have been proposed based on the selective swelling of hydrogels in response to pH by Sheppard, “Design of a Conductimetric Microsensor Based on Reversibly Swelling Polymer Hydrogels”, Transducers '91, 773-776 (1991) and Sheppard, et al., “Microfabricated Conductimetric pH Sensor”, Sensors and Actuators B, Vol. 28, pp. 95-102 (1995). Finally, sensor devices based on the selective swelling of hydrogels in response to glucose have been proposed by McCurley, “An Optical Biosensor Using A Fluorescent, Swelling Sensing Element”, Biosensors and Bioelectronics, Vol. 9, pp. 527-533 (1994) and Kikuchi, et al., “Glucose-Sensing Electrode Coated With Polymer Complex Gel Containing Phenylboronic Acid”, Anal. Chem., Vol. 68, pp. 823-828 (1996).

Sensor devices composed of a crystalline colloidal array polymerized in a hydrogel are disclosed in U.S. Pat. No. 5,898,004 of Asher et al. The sensor devices of Asher et al. require the use of a single film to detect and quantitate analyte concentration.

None of the art, however, discloses a colorimetric reagent as a chemical sensing device that utilizes a polymerized crystalline colloidal array (PCCA) which can be made into fragments and dispersed in a medium and be used as a detection means, as disclosed herein. The dispersion in a medium of PCCA allows for a simplified method for detecting analytes and allows for the detection of a chemical and/or biological species in the environment, for example from a chemical contamination or from a plume of chemical weapons in a battlefield, and also allows for remote sensing of chemical and/or biological species.

### SUMMARY OF THE INVENTION

The present invention is generally directed to a colorimetric reagent comprising a dispersion of fragments in a medium of a polymerized crystalline colloidal array (PCCA) wherein said PCCA is polymerized within a hydrogel. The hydrogel fragments undergo a volume change in response to specific chemical and/or biological species. Because the volume of the hydrogel fragments change, the lattice spacing of the crystalline colloidal array (CCA) embedded therein changes as well. The light diffraction properties of the CCA change as the lattice spacing is changed. Measuring the change in diffraction, therefore, indicates the presence or absence of the stimuli that causes the volume of the hydrogel to change. The diffraction from the polymerized crystalline colloidal array (PCCA) fragments of the colorimetric reagent of the present invention results in essentially a diffraction powder pattern. The powder pattern diffraction band edge shifts in proportion to analyte concentration. The present invention is also directed to methods for making and using this colorimetric reagent.

The colorimetric reagent of the present invention can be used to detect a number of specific stimuli. For example, it can be used to detect the presence of various chemicals, such as metal ions in solution and organic molecules such as glucose, making the reagent useful for chemical analysis. The colorimetric reagent can also be used to detect the presence of various gasses in and out of solution. As a biomedical detection device, the reagent can be used to detect the presence of antigens from various sources, and antibodies from various sources. Furthermore, the colorimetric reagent may be used for remote sensing of chemical and/or biological species in the environment, for example to detect a chemical contamination or to detect a plume of biological/chemical weapons at a distance in a battlefield. The PCCAs of the present invention which are capable of responding to a specific stimulus (such as a gas or a chemical or biological species) may be called intelligent PCCA (IPCCA).

One skilled in the art will appreciate that the various embodiments disclosed herein, as well as other embodiments within the scope of the invention, will have numerous applications in the environmental, medical, pharmaceutical, metallurgy, chemical and warfare fields.

It is thus an object of the present invention to provide a colorimetric reagent comprising a dispersion of fragments in a medium of a polymerized crystalline colloidal array (PCCA) sensing materials.

It is a further object of the invention to provide a colorimetric reagent that utilizes the light diffraction properties of a PCCA to detect the presence of various stimuli. For example, the colorimetric reagent of the present invention can detect the presence of, inter alia, chemicals (e.g. lead), gasses in solution, various medical conditions, biological molecules, and air born contaminants.

The present invention also provides a colorimetric reagent comprising a dispersion of fragments in a medium of a PCCA that swells in response to various stimuli, thereby changing the diffraction properties of the colorimetric reagent.

The colorimetric reagent of the present invention is useful, inter alia, in environmental applications, in the field of medical diagnostics, as a remote sensor device for detecting a plume of biological/chemical weapons at a distance in a battlefield, as a temperature sensor, as a pH sensor and as a lead sensor.

These and other objects of the invention will be more fully understood from the following description of the invention.

### BRIEF DESCRIPTION OF THE DRAWINGS

The present invention may be better understood with reference to the attached drawings of which

FIG. 1: (a) Face centered cubic crystalline colloidal array and (b) its diffraction spectrum.

FIG. 2: Diffraction from IPCCA particle dispersion. These randomly oriented diffracting particles give rise to rings of diffracted light similar to those obtained by X-ray powder diffraction. Back diffracted light, which has the longest diffraction wavelength, results from planes whose normal is parallel to the incident beam. Note that θ_{B} is the Bragg glancing incident angle.

FIG. 3: Model for detecting chemical/biological weapons. The colorimetric reagent is shot from the rocket at left as a mortar shell and explodes in the air above area of interest. The colorimetric reagent is now dispersed in the air. As it is floating down towards the ground a nsec pulsed beam of light is incident on it and a detector at a fixed angle from the light source measures the wavelength of light diffracted from the colorimetric reagent.

FIG. 4(a): Temperature dependence of diffraction spectrum of NIPAM IPCCA particle dispersion. The diffraction is measured in backscattering. As the temperature increases the wavelength blue shifts. (b): Temperature dependence of diffraction maximum for NIPAM IPCCA particles.

FIG. 5: (a) pH dependence of diffraction spectrum of IPCCA particles between pH 2.22-8.87. In this pH range the diffraction red shifts with increasing pH. (b): pH dependence of diffraction spectrum of IPCCA particles between pH 8.87-11.11. The diffraction is measured in backscattering. Above pH 10 all carboxylates are ionized and the pH increase only increases the ionic strength, which blue shifts the diffraction. (c): pH dependence of diffraction.

FIG. 6: (a) Pb²⁺ concentration dependence of diffraction spectrum of IPCCA particles. The diffraction red shifts as the Pb²⁺ concentration increases. (b): Pb²⁺ concentration dependence of diffraction maximum.

### DETAILED DESCRIPTION OF THE INVENTION

The colorimetric reagent of the present invention generally comprises fragments of a polymerized crystalline colloidal array (PCCA) comprising a hydrogel which undergoes a volume change in response to a specific stimuli, such as a chemical and/or biological species and a light diffracting crystalline colloidal array (CCA) of charged particles polymerized in the hydrogel, the crystalline colloidal array having a lattice spacing that changes when the volume of the hydrogel changes thereby causing the light diffraction of said crystalline colloidal array to change. Instead of requiring the use of a single film, the reagent utilizes a dispersion of the PCCA in a medium and the diffraction from this dispersion is used to determine the concentration of analyte. The colorimetric reagent comprises fragments of optical, hydrogel based sensors of U.S. Pat. No. 5,898,004 of Asher et al. (incorporated herein by reference) that combine the light diffraction properties of crystalline colloidal arrays (CCA) with the conformational changes that various polymers undergo in response to external stimuli.

As used herein, the term medium may include, inter alia, a surrounding environment, such as, but not limited to, an aqueous solution, a solvent (such as an organic or inorganic solvent), a gas (e.g. air), etc.

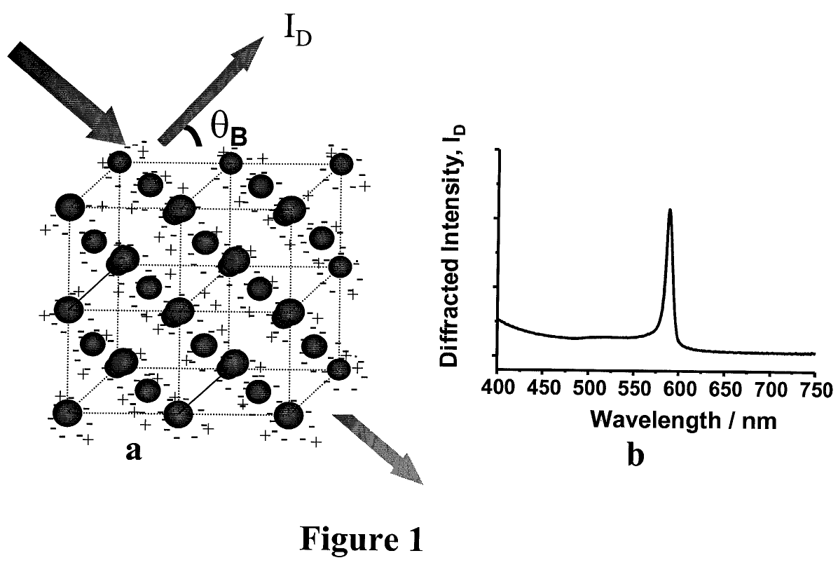

Monodisperse, highly charged colloidal particles dispersed in very low ionic strength liquid media self-assemble due to electrostatic repulsion to form CCA. These ordered structures are either body-centered cubic (BCC) or face-centered cubic (FCC) arrays with lattice constants in the mesoscale range (50-500 nanometers (nm)) (see U.S. Pat. No. 5,898,004 of Asher et al., incorporated herein by reference). A face-centered CCA is shown in FIG. 1 along with its typical CCA diffraction spectrum. Just as atomic crystals diffract x-rays meeting the Bragg condition, CCA, such as those depicted in FIG. 1, diffract ultraviolet (UV), visible and near infrared (NIR) light. CCA can be prepared as macroscopically ordered arrays from non-close packed spheres. Such arrays exhibit highly efficient Bragg diffraction; nearly all light meeting the Bragg condition is diffracted, while adjacent spectral regions not meeting the Bragg conditions will freely transmit. “Non-close packed spheres” refers to an ordering wherein the spheres are spaced by some distance from each other. The Bragg diffraction law is represented by the following formula:

mλ=2ndsin θ;

where m is the order of diffraction, λ is the wavelength of incident light, n is the suspension refractive index, d is the interplanar spacing, and θ is the angle between the incident light beam and the crystal planes.

Some polymers reversibly change conformation and volume in response to a specific external stimulus. For example, almost all polymers undergo some reversible conformational change and volume change with changes in solvents, and some, such as poly N-isopropylacrylamide (PNIPAM), undergo conformational changes in response to temperature changes. Solutes that interact with the side groups on the polymer backbone may also induce conformational changes. For example the introduction of ionized groups onto the backbone of the polymer may make the polymer sensitive to changes in ionic strength. Polymers that change conformation in response to the concentration of a single, specific solute can therefore be prepared by adding to that polymer a functional group that selectively interacts with that single solute. Volume changes between 0.1 and ˜300%, or even greater, are contemplated by the present invention. The volume response exhibited by these hydrogels allows for their broad application in areas including but not limited to chemomechanical systems, separation devices and sensor devices.

These PCCA may be used as a sensor device (see U.S. Pat. No. 5,898,004). The present invention relates to a colorimetric reagent comprising fragments of PCCA. The PCCA of the colorimetric reagent comprises a hydrogel characterized by the property of undergoing a volume change in response to a specific chemical and/or biological species; and a light diffracting crystalline colloidal array (CCA) of charged particles polymerized in the hydrogel; the crystalline colloidal array having a lattice spacing that changes when the volume of the hydrogel changes, thereby causing the light diffraction of the crystalline colloidal array to change. The hydrogel generally comprises a crosslinking agent, a monomer component and a molecular recognition component. The crosslinking agent can be any crosslinking agent compatible with the other components of the hydrogel. Suitable crosslinkers include, inter alia, N,N′-methylenebisacrylamide, methylenebismethacrylamide and ethyleneglycoldimethacrylate, with N,N′methylenebisacrylamide being preferred. In addition to forming the polymer network in the CCA, the cross-linking agent assists formation of the hydrogel and strengthens the resulting hydrogel film so that a self-supporting film results. Hydrogel films can be formed when as little as 1 part crosslinker in 100 parts by weight of the monomer is used. Generally, increasing the amount of crosslinking agent lowers the sensitivity of the hydrogel to the analyte being detected. Preferably, crosslinker is used in an amount between about 4 and 15% of monomer weight, more preferably about 5% of monomer weight.

The hydrogel monomer component of the hydrogels of the present invention can be any compound that forms a hydrogel that undergoes a volume change in response to a stimulus or stimuli. Examples of suitable gels include, but are not limited to, acrylamide gels, purified agarose gels, N-vinylpyrolidone gels and methacrylate gels. Preferred hydrogel monomer components for use in the present invention are acrylamide (AMD) and N-isopropylacrylamide (NIPAM).

The phase transition properties of the hydrogel are modified by functionalizing the hydrogel with a reagent that specifically binds an analyte of interest. Thus, the hydrogel is modified so as to detect the presence of an analyte by means of this molecular recognition component. More specifically, a monomer capable of selectively interacting with a specific solute is incorporated in the hydrogel. Typically, the higher the molecular recognition component concentration, the more sensitive will be the colorimetric reagent of the present invention to the desired analyte. This relationship, however, is only observed up to a certain concentration of the molecular recognition component, after which the sensitivity of the hydrogel decreases. Any monomer having molecular recognition capabilities for the desired analyte can be used. For example, 4-acrylamidobenzo 18-crown-6 ether, which selectively binds Group I cations and preferably binds Pb²⁺ ions, can be used if Pb²⁺ is the analyte of interest. Other crown ethers, cyclodextrans, calixarenes, and other chelating agents can also be used. In addition, the monomer may be n-isopropyl acrylamide, which can allow for sensitive temperature change detection.

When the analyte binds to the hydrogel matrix, it causes a change in the hydrogel matrix, and therefore changes the swelling properties of the hydrogel. As the hydrogel shrinks and swells, the CCA embedded in the hydrogel follows. As the CCA changes dimension, the resulting diffraction wavelength alteration reports on the array volume change. The diffraction shifts to shorter wavelengths as the hydrogel shrinks, and to longer wavelengths as the hydrogel swells. Measuring this alteration, therefore, allows for detection of the analyte, which caused the volume change.

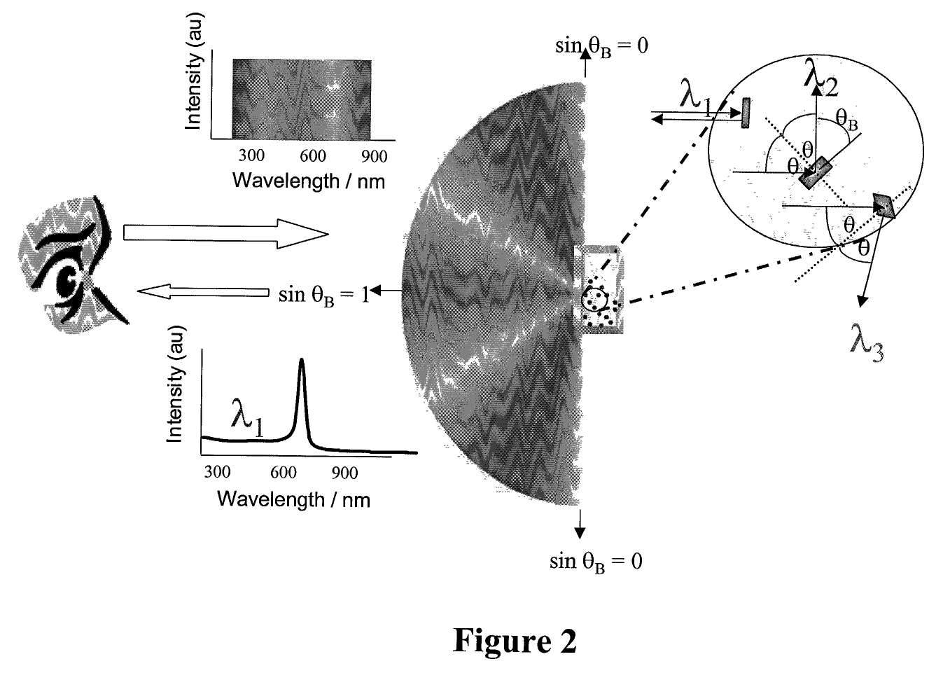

The colorimetric reagent of the present invention comprises a dispersion of the PCCA in a medium and uses the diffraction pattern from the dispersion to determine the concentration of analyte. The operative principle is that the diffraction from the PCCA changes due to binding of analyte. The diffraction from the PCCA fragments in the dispersion results in essentially a diffraction powder pattern. Polychromatic light meeting the Bragg condition will be dispersed with the longest wavelength meeting the Bragg condition diffracted at the largest angle (θ_{B}=90°, FIG. 2). In FIG. 2, at sin θ_{B}=1(180°−2 θ_{B}=0°) a back diffracted red beam (the longest wavelength) is obtained. As sin θ decreases, the diffraction shifts to shorter wavelengths. See FIG. 2.

The novel colorimetric reagent of the present invention can be used as a simple liquid reagent, which allows a color test to be used for determination of an analyte concentration. Either colorimetric reagent may be added dropwise to the solution of interest or the solution of interest may be added to the dispersion of hydrogel fragments. The colorimetric reagent of the present invention may simplify detection of analytes. Analyte quantitation may be achieved, for example, using either a reflection or transmission spectrophotometer to determine the band edge. In addition, the colorimetric reagent of the present invention may also be useful for detecting analyte in the environment. For example it may be used as a remote sensor device to detect a chemical contamination in the environment or a plume of biological/chemical weapons at a distance in a battle field.

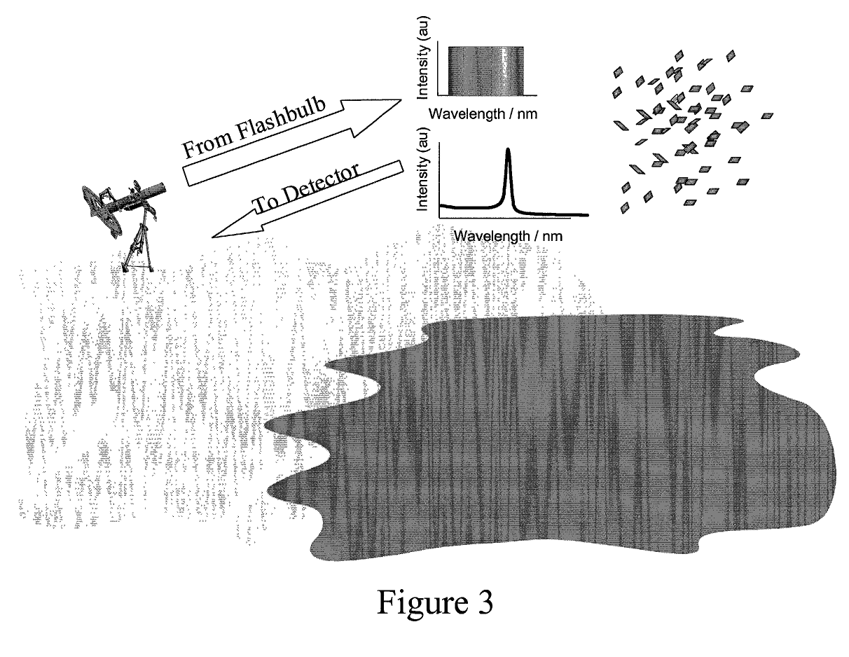

The use of the colorimetric reagent as a remote sensor device for the detection of the presence of a biological/chemical weapons or a contamination in the environment could be achieved, for example, by firing a rocket, or a mortar shell in the distance which releases a solution containing fragments of PCCA in water droplets. A light source such as a flash lamp could be used to illuminate the PCCA fragments and a color camera would record the color of the powder diffraction pattern. The position of the band edge would indicate the concentration of the analyte, which partitioned into the droplets of water released by the rocket or mortar shell (See FIG. 3). This system is useful to detect a chemical specie in the environment in the day as well as in the night since the nsec pulsed flashlamp/flashbulb excitation can be detected with a telescope which would use time gating in order to reject illumination from the sun. The colorimetic reagent needed for the detection of biological/chemical weapons would comprise a molecular recognition component sensitive to a biological/chemical weapon. One molecular recognition component that is useful is acetylcholinesterase, which binds many phosphorous containing chemical weapons.

As stated above, the colorimetric reagent of the present invention combines CCA technology with modified hydrogels, creating a PCCA, which may be in a dispersion to provide a colorimetric reagent useful, for example, as a sensor of analyte in solution or in the atmosphere. More specifically, a hydrogel having the characteristics described above is polymerized around a fluid CCA that is then made into fragments achieved by using a tissue homogenizer (Biospec Products Inc. Model 985370) or a cell homogenizer. The film can also be frozen and then pulverized in a mortar and pestle. Changes in the volume of the hydrogel matrix change the lattice spacing of the embedded CCA, thus changing the color of the light diffracted. The colorimetric reagent is well suited for analyte sensor applications due to the unique ability to directly measure the volume change of the hydrogel by monitoring the diffraction wavelength from the CCA. In many applications, the change in color can be detected by the unaided eye. For example, a less than 5% expansion in volume can yield a color change detectable by the unaided eye.

Detection of an analyte in the atmosphere is useful, for example, for detecting atmospheric contamination, which may be achieved at a distance. The ability to detect atmospheric contamination from a distance may allow for the early warning of a community to the existence of an atmospheric contamination. The detection of an atmospheric contaminant may be achieved using a PCCA comprising a molecular recognition component, which is sensitive to the atmospheric contaminant.

A method for making a colorimetric reagent according to the present invention generally comprises the steps of allowing monodisperse, charged colloidal particles to self assemble into a crystalline colloidal array; adding a first comonomer that is a hydrogel monomer, a crosslinking agent, a second comonomer that is a molecular recognition component to a medium comprising said crystalline colloidal array and a polymerization initiator; and polymerizing the mixture to form a crystalline colloidal array embedded in a hydrogel. The resulting crystalline colloidal array embedded in the hydrogel is then made into fragments by use of a cell homogenizer. The fragments are dispersed in a medium, for example water, to create the colorimetric reagents of the present invention.

An alternative method for making a colorimetric reagent according to the present invention generally comprises the steps of allowing the monodisperse, charged colloidal particles to self assemble into a crystalline colloidal array; adding a crosslinking agent, a gel monomer, and a polymerization initiator; polymerizing the mixture to form a crystalline colloidal array embedded in a hydrogel; adding a molecular recognition component capable of binding with the hydrogel, and making fragments of the resulting CCA embedded in a hydrogel which are then dispersed in a medium as described above and in Examples 2-4 below.

Any suitable colloidal particles can be used. For example, the particles used to create the CCA can be colloidal polystyrene, polymethylmethacrylate, silicon dioxide, aluminum oxide, polytetrafluoroethylene or any other suitable materials, which are generally uniform in size and surface charge. Colloidal polystyrene is preferred. The particles are chosen depending upon the optimum degree of ordering and the resulting lattice spacing desired for the particular application. The particles preferably have a diameter between about 50 and 1000 nanometers and may be either synthesized as discussed below or obtained commercially. Colloidal particles that can be used in accordance with this embodiment have been described by Reese et al., 2000, Journal of Colloid and Interface Science 232: 76-80, incorporated herein by reference.

Monodisperse colloids can be prepared by emulsion polymerization or any other means. For example, an emulsion polymer colloid can be prepared by mixing the desired monomer with a cross-linking agent, a surfactant to aid in the formation of the emulsion, a buffer to keep the pH of the solution constant and to prevent particle coagulation, and a free-radical initiator to initiate polymerization. In a preferred embodiment, the monomer is styrene, the cross-linking agent is divinylbenzene, the surfactant is sodium-di(1,3-dimethylbutyl)sulfosuccinate, the initiator is preferably ammonium persulfate and an ionic comonomer is also added, preferably 1-sodium, 1-allyloxy-2-hydroxypropane sulfonate. Other suitable compounds can also be used to prepare the emulsion polymer colloid, so long as compatibility problems do not arise. The particles should then be purified by the use of centrifugation, dialysis and/or an ion exchange resin.

Following polymerization, the particles may be stored in an ion exchange resin, preferably in a bath of 10% by weight suspension of ion exchange resin such as analytical grade AG501-X8 mixed bed resin commercially available from Bio-rad of Richmond, Calif. The ion exchange resin should preferably be cleaned prior to use through a suitable procedure such as that of Vanderhoffet al., 1968, Journal of Colloid and Interface Science, 28, 336-337.

The electrically charged particles are then allowed to self assemble to form a crystalline colloidal array. This assembly takes place in a suitable solvent, preferably water. A hydrogel monomer, a molecular recognition component, a cross-linking agent and a polymerization initiator are then added to the CCA. Any suitable initiator can be used, such as a thermal initiator or a photoinitiator. Preferably, a UV photoinitiator is used. A preferred UV photoinitiator for this use is 2,2′-diethoxyacetophenone. Any cross-linking agent, gel monomer and molecular recognition component discussed above can be used.

After formation, the mixture is then polymerized. Any means known in the art can be used to initiate polymerization, so long as the method for polymerization does not destroy or otherwise disorder the CCA. Preferably, the polymerization is accomplished by placing the mixture between two plates, preferably quartz plates separated by a parafilm spacer, at a temperature from between about 0° to 10° C. The plates are then exposed to UV light. Exposure to the UV light effects complete polymerization after about 5 minutes. Upon completion of the polymerization, the plates are removed and a stable polymerized CCA (PCCA) results. This film can be approximately 150 microns thick and can be made thinner or thicker based upon the needs of the user.

In a preferred embodiment, the hydrogel is composed of a copolymer of acrylamide (AMD) and 4-acrylamidobenzo 18-crown-6 ether crosslinked with N,N′-methylenebisacrylamide. The crown ether in the hydrogel complexes with metal cations, with an affinity that depends both on the ability of the cation to fit into the cavity of the crown ether, and the charge of the ion. The AMD gel is hydrophilic. The copolymer is highly sensitive to slight changes in its charge state due the complexation of small amounts of cations. As the crown ether binds to cations, the entire copolymer becomes a polyelectrolyte gel causing the PCCA to swell to minimize the free energy of the system and the diffraction wavelength increases.

In addition, the present invention contemplates embodiments in which the gel monomer will change volume in response to temperature changes. For example, NIPAM hydrogels change volume with changes in temperature. Temperature has a large effect on the diffraction of the sensor. The diffraction wavelength of the sensor is 625 nm at 7° C., decreases to 555 nm at 23° C., and further decreases to 440 nm at 34° C.

Therefore, the colorimetric reagent of one preferred embodiment of the present invention is particularly useful as a temperature sensor. See Example 4 below. Hydrogels containing NIPAM show a temperature induced reversible volume phase transition from a swollen to a collapsed state. See Weissman et al., 1996, Science 274:959; Tanaka, 1992, Nature 355:430-432; Mafe et al., 1997, Phys. Rev. Lett. 79:3086-3089; Hirokawa and Tanaka, 1984, J. Chem. Phys. 81:6379; Shibayama and Tanaka, 1995, J. Chem. Phys. 102:9392; English et al., 1997, J. Chem. Phys. 107:1645-1654; English et al., 1998, Polymer 39:5893-5897, all incorporated herein by reference. It has previously been demonstrated that a N-isopropylacrylamide (NIPAM) IPCCA single crystal could be used as a temperature sensor. See Weissman et al., 1996, Science 274:959, incorporated herein by reference. When NIPAM IPCCA are exposed to cold water, they swell, but when the temperature increases they undergo a reversible volume phase transition to a collapsed state with a resulting diffraction blue shift.

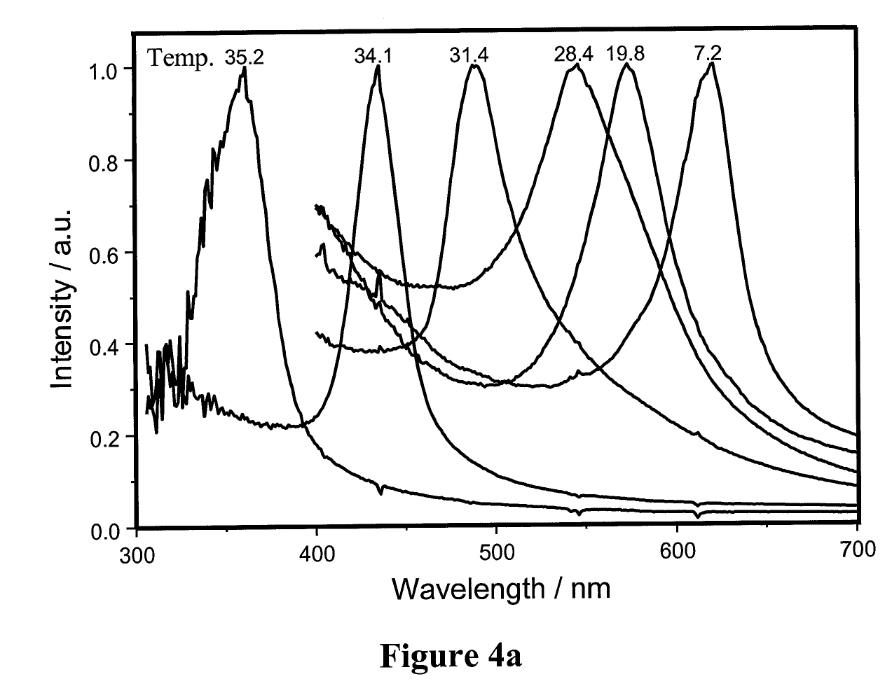

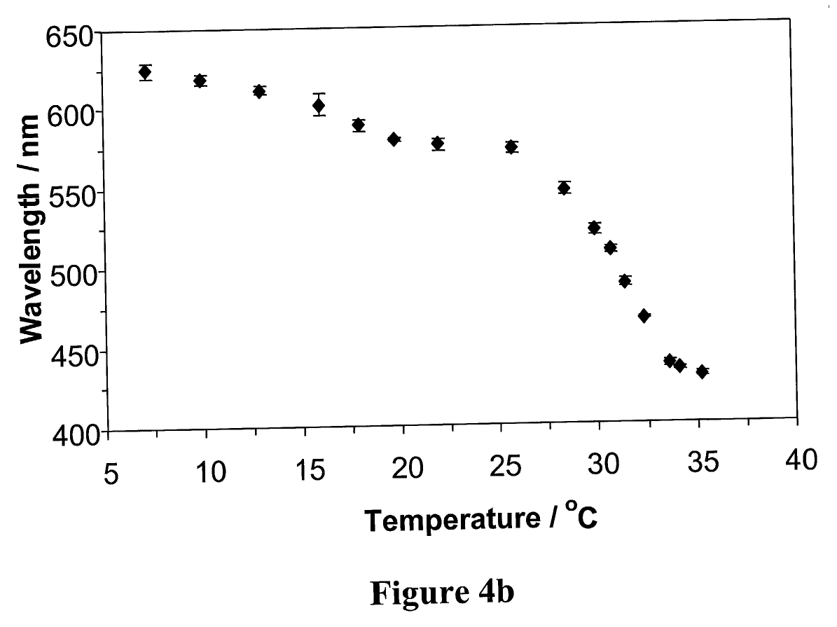

FIGS. 4a and 4b show that NIPAM IPCCA particles show similar diffraction temperature dependencies. At 7.2° C. the particles diffract 625 nm light, while at 35.2° C. the particles diffract 425 nm light. Between 7.2° C. to 25° C. the diffraction shifts from 625 nm to 560 nm, which gives a sensitivity of ˜3 nm/° C. A more sensitive response occurs around the phase transition temperature, with an 80 nm shift occurring over the 6 degree range of 27.5° C. to 33.5° C., giving a sensitivity of ˜17 nm/° C. Since the likely error for determining the diffraction wavelength maximum is ˜1 nm, in a preferred embodiment of the present invention, the IPCCA particles resolve temperature differences of <0.5° C. away from the phase transition temperature and more preferably <0.05.° C. around the phase transition temperature.

The phase transition temperature may be varied by altering the hydrogel chemical composition. For example, adding acrylic acid to the hydrogel backbone shifts the phase transition temperature to ˜50° C. See Shibayama and Tanaka, 1995, J. Chem. Phys. 102:9392. Thus, the present invention provides colorimetric reagent temperature sensors useful in the temperature range from about 0° C. to about 60° C. Further, the present invention is also useful for detecting temperature gradients. For example, the particles could be dispersed in a vessel of water and a telescope could be used to direct light into the tank and to image the light diffracted from a certain volume element. The colorimetric reagent, when used as a temperature sensing device may detect changes in any environment when exposed thereto.

The IPCCA colorimetric reagent of the present invention may also be useful as a gas sensing device and may be used for detecting gases in solution. In this embodiment of the invention, the colorimetric reagent may comprise a molecular recognition component, such as a gas binding component, which may be bound by conventional means (as discussed above using a linking molecule) to the hydrogel of the colorimetric reagent. The gas binding component of the colorimetric reagent of the present invention can bind to a gas and, upon binding, the hydrogel will undergo a volume phase transition. When the hydrogel undergoes the volume phase transition, the CCA, which is embedded in the hydrogel matrix, will also undergo a change in volume. This in turn will cause a change in the wavelength of light diffracted from the colorimetric reagent fragments. By binding a gas binding component to the hydrogel the colorimetric reagent will be able to sense dissolved amounts of gas (e.g., water vapor; oxygen) in solution. In a preferred embodiment, the gas binding component is glucose oxidase. When glucose oxidase was bound to the hydrogel and placed in a solution with a known glucose concentration, the hydrogel became sensitive to the amount of dissolved oxygen in the solution. See Holtz et al., Anal Chem, 1998, 70:780-791. In another embodiment of the invention, the colorimetric reagent comprising a gas binding component may be placed in a container with a semi permeable membrane that will allow only certain gas molecules to pass through it. The semipermeable membrane blocks certain molecules from entering into the hydrogel, while allowing others to enter and be sensed by the colorimetric reagent. This aids in the selectivity and sensitivity of the colorimetric reagent comprising a gas binding component toward a particular gas.

In addition, the colorimetric reagent of a further preferred embodiment of the present invention is particularly useful as a pH sensor. See Example 3 below. An IPCCA sensing film has recently been developed that can determine pH and ionic strength. See Lee and Asher, 2000, Am. Chem. Soc. 122:9539, incorporated herein by reference. This IPCCA is a hydrolyzed acrylamide hydrogel in which some amide groups are converted to carboxylic acids. The pH dependence is due to the acid-base equilibria of the carboxyl groups. As the carboxyl groups ionize, anions become localized on the hydrogel. This causes a Donnan potential resulting from a difference in the chemical potential between the hydrogel and the medium surrounding the hydrogel causing an osmotic pressure difference, which swells the IPCCA and causes the diffraction wavelength to red shift.

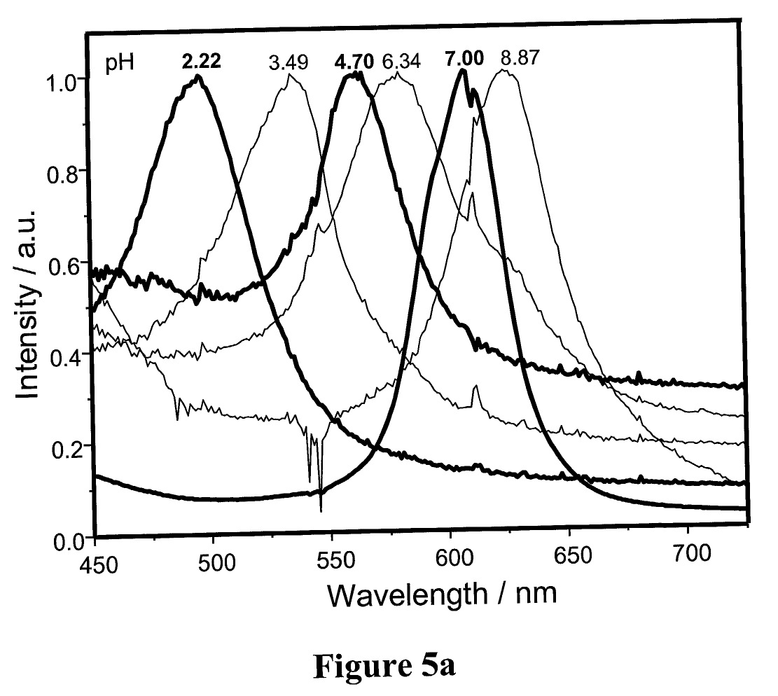

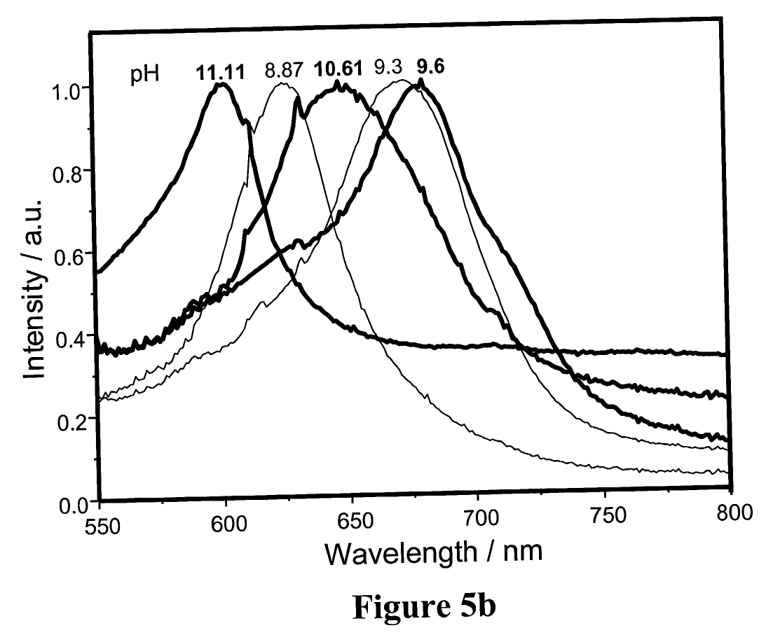

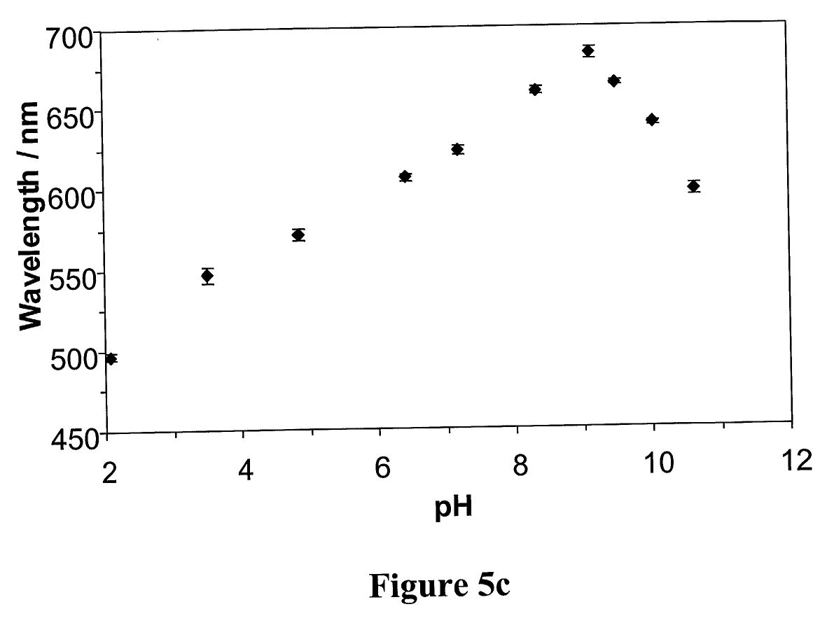

FIGS. 5a, 5b, and 5c show the pH dependence of diffraction for the colorimetric reagent of the present invention. The diffraction monotonically red shifts from 500 to 655 nm between pH 2 to 9.6, whereupon it blue shifts from 655 nm to 590 nm between pH 9.6 to 11.11. The blue shift results from the decrease in osmotic pressure due to the increased solution ionic strength at the high pH values. The response is relatively linear up to a pH of 9.6 with a sensitivity of 20 nm/pH unit. Given a 1 nm resolution we can determine pH with a 0.05 pH unit resolution in deionized water.

For solutions with a defined ionic strength, the colorimetric reagent of the present invention can be calibrated to determine pH. Ionic strength can independently be determined by utilizing IPCCA with ionic groups that do not undergo pH titrations. Thus, two separate IPCCA colorimetric reagents can be used in parallel. Ionic strength can be determined and then an ionic strength calibrated pH sensing IPCCA can be made to determine the solution pH. Alternatively, the ionic strength may be determined by any means known in the art.

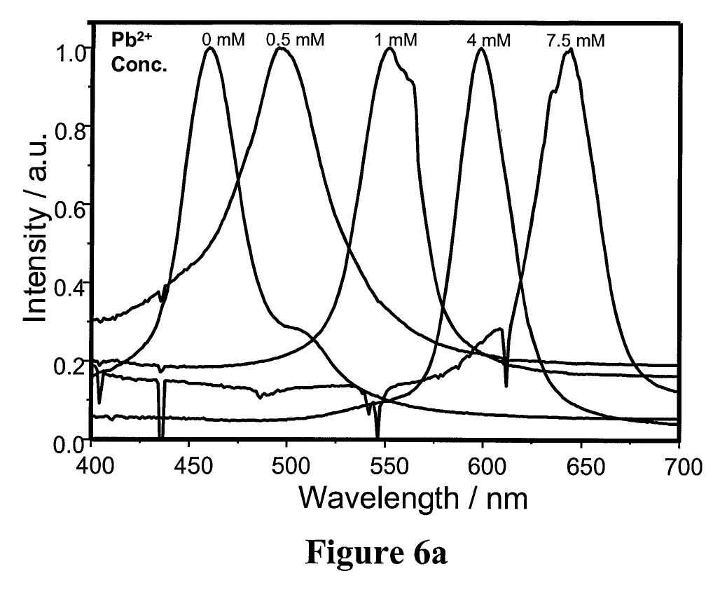

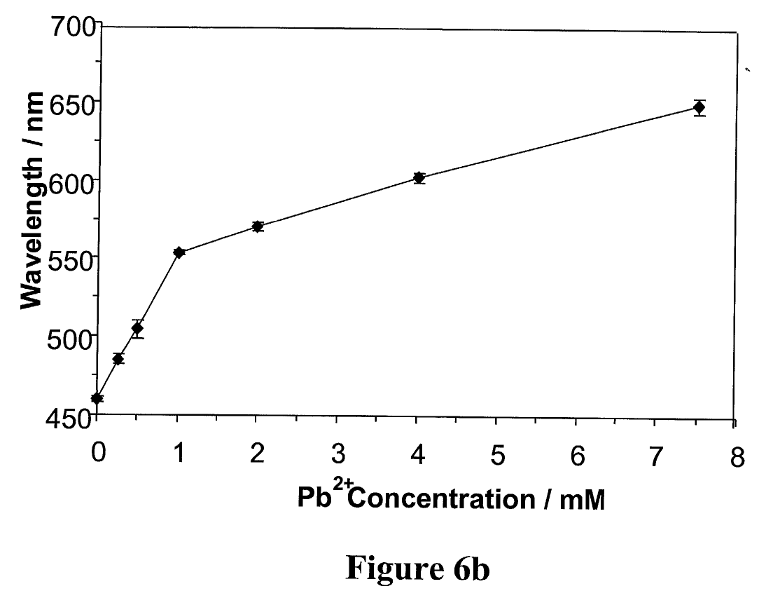

Another preferred embodiment of the present invention is a colorimetric reagent useful as a sensor for lead. The detector swells in lead concentrations between about 2 μM and about 10 mM. FIG. 6a and 6b show the diffraction pattern of a colorimetric reagent of the present invention in response to a variation in lead concentration. The diffraction pattern of the lead-detecting solution is dependent upon the lead concentration. The solution has a blue appearance in the absence of lead. However, the addition of 0.5 mM and 7.5 mM lead shifts the color to blue-green and red, respectively. This detector functions as an easy to use, sensitive detector that is blue at lead concentrations of about 50 μM or less and green at concentrations of about 300 μM. The color change of the colorimetric reagent can be detected by the unaided human eye at lead concentrations of approximately 200 μM, and can be detected by a spectrophotometer at even lower concentrations. At concentrations higher than about 20 mM, the fragments of PCCA in the colorimetric reagent of the present invention shrink and the wavelength diffracted moves to shorter wavelengths.

Alternatively, the incorporation of other crown ethers in the hydrogel produces a sensor that selects other cations. The selectivity of the sensor is limited by non-selective binding of the crown ether with other cations. Similarly, use of functionalized compounds, other than crown ethers, such as cyclodextrans, calixarenes, or other chelating agents, can produce colorimetric reagents that respond to still other stimuli.

The response rate of the fragments of IPCCA having an area of about 250 square microns which are dispersed in the colorimetric reagent of the present invention, as described above, is preferably less than about 10 minutes and more preferably less than about 5 minutes. The response rate may be improved by decreasing the size of the IPCCA fragments. The response rate is partially determined by the mass transport of cations into the gel, and partially determined by the kinetics of complexation. Decreasing the fragment size and the monomer content of the gel may markedly increase the rate of analyte mass transport to the active sites on the gel, and therefore decrease response time. The response rate may also be affected by the molecular recognition component used, as some may be more selective than others. Response rates of between about 1 and 5 minutes can be achieved with 250 square micron fragments and response rates on the order of seconds can be achieved with smaller fragments. The response rate is inversely proportional to the size of the fragments.

In another embodiment of the present invention, the hydrogel in which the CCA is polymerized comprises a crosslinking agent, a hydrogel monomer and a biomolecular recognition component. This biomolecular recognition component is a biomolecule that selectively binds a specific chemical and/or biological species as part of its biological function. This component can be bound to the hydrogel directly or by one or more linking molecules. Examples of such biomolecular recognition components include, but are not limited to, enzymes, antibodies, antigens, porphyrins, ferritin, or pheromone receptors. These natural recognition components can respond to simple chemical and/or biological species, or to the presence of a biological species, such as particular proteins, DNA, RNA, microorganisms (such as virus, bacteria and yeast), etc. The PCCA fragments dispersed in the colorimetric reagent of the present invention can therefore further comprise one or more linking molecules that bind the biomolecular recognition component to the hydrogel monomer. In addition, the biomolecular recognition component can be modified by being reacted with a molecule that can be bound to the linking agent, or to the hydrogel itself. A particularly preferred linking molecule is 5-(biotinamido)pentylamine, and a preferred molecule for reaction with the biomolecular recognition component is avidin; avidin is a protein extracted from egg whites and has four binding sites for biotin. The colorimetric reagents of this embodiment have particular application in the area of detection of disease markers, for example in detecting the presence of HIV antibodies, and for detecting chemical and biological weapons. The hydrogel can be sensitive to very low concentrations of species, if the recognition element has a high binding constant. This is attributable to the fact that the PCCA recognition element concentrates the analyte within the PCCA.

For example, an antigen can be added to a hydrogel monomer to form a hydrogel that binds such things as antibodies to tuberculosis cells, cancer cells, or HIV. The antigen is chosen based on what medical condition is to be detected. Enzymes can also be bound to the gel for medical diagnostics and for biological and chemical weapon detection. For example, binding glucose oxidase to the hydrogel will allow for the detection of glucose. Thus this embodiment of the present invention has application as a medical diagnostic tool and chemical and biological warfare tool. As above, the sensitivity of the sensor can be adjusted to the desired concentration by modifying the ratio of hydrogel monomer to recognition component, the degree of crosslinking and the hydrophobicity of the hydrogel monomer. Hydrophobicity can be adjusted with the addition of another monomer that is either more or less hydrophobic than the hydrogel monomer, depending on the needs of the user.

The antibody and antigen based sensors function much the same way as the chemical sensors discussed above. That is, the gel volume changes when the hydrogel becomes bound to a chemical specie that changes the free energy of the hydrogel.

In the case of the enzyme-based sensors, the enzyme may change the chemical nature of the analyte by first binding to the analyte substrate and then cleaving or otherwise reacting with it. This binding or reaction results in a change in the charge bound to the hydrogel. The hydrogel of the enzyme based sensor changes volume because of the resulting change in the bound charge concentration in the interior of the hydrogel and the changes in the concentration of mobile counterions. This causes an osmotic pressure imbalance between the inside and outside of the gel. A solvent, preferably water, diffuses into the hydrogel to relieve that pressure imbalance; it is this excess solvent that causes the hydrogel to swell. If the hydrogel is washed in pure solvent, water, the sensor returns to its previous volume and can be reused. The response of the sensor, therefore, is dependent upon the concentration and amount of substrate in its immediate environment.

The colorimetric reagents which can be used as medical diagnostic and biological/chemical weapon detection tools can also be made by polymerizing a CCA in a hydrogel comprising a crosslinking agent and a gel monomer such as those described above. Following formation of the PCCA, a molecular recognition component is added. In the preferred embodiment, addition of the molecular recognition component is accomplished by first hydrolyzing the PCCA. Any means known in the art can be used to effect hydrolysis; a preferred method is to place the PCCA in a 0.1M solution of sodium hydroxide for about 10 minutes. Hydrolysis of the PCCA serves to establish acidic, reactive sites on the PCCA matrix. Preferably, the hydrolysis is a partial hydrolysis in which 10 to 30% of the amide groups on the PCCA matrix are hydrolyzed to carboxylic acid groups. This is accomplished by hydrolyzing for about 10 minutes. The hydrolyzed PCCA is then reacted with a linking molecule and a coupling agent that binds the compound to the carboxylic acid groups on the matrix. A preferred linking molecule is a 5-(biotinamido)pentylamine and a preferred coupling agent is 1-(3-dimethylaminopropyl)-3-ethylcarbodiimide. Other compounds and water-soluble coupling agents can also be used. As will be appreciated by one skilled in the art, the reaction can be performed without a coupling agent, but proceeds more rapidly in the presence of one. The PCCA should be reacted with the linking molecule for a period of time sufficient to effect reaction of all of the acid groups; when using a coupling agent this is typically between about 3 to 6 hours. A molecular recognition component, such as an enzyme, antibody or antigen, is then added, and binds to the compound. The molecular recognition component is first bound to a compound having an affinity for the linking molecule. A preferred compound for this use is avidin, which is preferred when using biotin as the linking molecule. Thus the enzyme is bound to the PCCA without destroying the CCA structure or the reactivity of the enzyme. The resultant PCCA is then made into fragments as described above and dispersed into a medium. The enzymes then react with a specific compound. For example, if glucose oxidase is used as the enzyme, the gel will cleave glucose, and if beta-D-galactosidase is used as the enzyme the gel will cleave beta-D-galactose.

As will be appreciated by those skilled in the art, the biomolecular recognition component can be added in numerous ways. In the preferred embodiment described above, this addition is effected by reacting the biomolecular recognition component with avidin, which binds to the 5-(biotinamido)pentylamino bound to the gel matrix. This method, therefore, essentially uses two linking molecules. Embodiments using only one linking molecule or more than two linking molecules, as well as no linking molecule at all, are also within the scope of the invention.

In yet another embodiment of the present invention, interpenetrating networks (IPNs) can be used to produce sensors with recognition elements that are normally incompatible with the required self assembly of the CCA prior to polymerization into a PCCA. For example, some molecular recognition functionalized comonomers may be ionic, and would screen the electrostatic colloidal particle repulsive interactions required for CCA self assembly. In this case, the PCCA is made in two steps. First, a loosely crosslinked PCCA is formed without the molecular recognition component. Following formation of this PCCA, a second monomer having the recognition component is diffused into the existing PCCA network. A second polymerization is then effected, wherein the polymer chains of the second network will form an interpenetrating network within the voids of the first network. The second network of the IPN will shrink or swell in response to the presence of the analyte, and the PCCA will expand or contract along with the second network due to the physical entanglement of the two networks. The IPN can then be made into fragments and dispersed in a medium, as described above.

In addition to using the colorimetric reagents of the present invention for the sensing of analytes in water, the hydrogel fragments can also be dispersed in other mediums, such as solvents and gases. Nonlimiting examples of solvents include alcohol solutions (e.g., methanol, ethanol, isopropanol, etc.); aromatic solvents (e.g., toluene, benzene, styrene); and other solvents (e.g., acetone and dimethyl sufoxide(DMSO)). Nonlimiting examples of gases include air in the environment. In order to place the hydrogel into a medium other than water, it is necessary to slowly replace the water with the new medium. It is recommended, for example, when placing the hydrogel into DMSO that the water is replaced with DMSO in small increments, allowing the hydrogel to equilibrate with each increase in DMSO until a 100% solution of DMSO is obtained. For solvents that are not miscible with water such as benzene, it is recommended to place the hydrogel into an acetone/benzene solution. The water will partition out of the hydrogel into the acetone phase. Once the water is eliminated from the hydrogel, the hydrogel can be placed into a 100% benzene solution. The hydrogel can respond to analytes in these solvents as it did in water. In one embodiment of the invention, the colorimetric reagent comprises a molecular recognition component such as β-cyclodextrin which may be bound to the hydrogel of the colorimetric reagent as discussed above using a linking molecule. This molecular recognition component can bind organic molecules, in particular polyaromatic hydrocarbons. This sensor has been tested in both water and DMSO for the ability to sense 2-napthalene sulfonic acid (NSA). The response to NSA is similar in water and DMSO, with the DMSO system being more responsive than the water system. The factors that govern the response of the colorimetric reagent in water are the factors that govern the response in other solvents. The actual wavelength shifts may vary because there is some solvent dependence to the wavelength shift.

Methods of using the above colorimetric reagents for detecting the concentration of a selected chemical specie are also provided. Following polymerization of the CCA in the hydrogel, producing fragments of the PCCA and dispersing the fragments into solution, these methods of use further include the steps of measuring the diffracted wavelength of said fragments dispersed in a medium; contacting said fragments with analyte; measuring the diffracted wavelength of said fragments following exposure to said analyte; and comparing the change in diffracted wavelength to determine concentration of said analyte. As discussed above, when a stimulus, such as a chemical specie, becomes bound to the hydrogel, thereby causing the volume of the hydrogel to change, the lattice spacing of the CCA also changes. Accordingly, the diffracted wavelength of the CCA changes as the volume of the hydrogel changes. By determining the change in diffracted wavelength, the volume change of the hydrogel and, therefore, the concentration of the chemical specie can be determined. The higher the concentration of the chemical specie, the greater the swelling volume of the gel. The diffraction from the PCCA fragments results in essentially a powder pattern for the diffraction. Instead of a single shift of the diffraction band that is observed from a single film at a particular incident and diffraction angle, the powder pattern diffraction band edge shifts in proportion to the analyte concentration. Polychromatic light meeting the Bragg condition will be dispersed with the longest wavelength meeting the Bragg condition diffracted at the largest angle (θ_{B}=90°, FIG. 2). In FIG. 2, at sin θ_{B}=1 (180°−2θ_{B}=0°) a back diffracted red beam (the longest wavelength) is obtained. As sin θ decreases the diffraction shifts to shorter wavelengths. By fixing the angle between the incident beam and the detector, only a small diffraction angular width is monitored. FIG. 2 illustrates a system where the detector and the incident beam are colinear, such that sin θ_{B}=1. When these particles shrink or swell in response to environmental changes, the diffraction wavelength shifts.

The change in diffracted wavelength can be determined by using instrumentation, such as a spectrometer or a spectrophotometer, such as a reflection or transmission spectrophotometer. In many cases, the diffracted wavelength change can also be seen by the unassisted human eye because the PCCA dispersion of the colorimetric reagent will change color or by using a flashlamp or flashbulb.

The present invention further provides a remote sensor device. The remote sensor device allows for the reading of temperature or analyte concentration from a distance. For example, the colorimetric reagent of the present invention may be placed in a certain location for local pH, temperature, or analyte detection of the environment in that location. The monitoring of the pH, temperature or analyte may be carried out from a distance using a monitoring means, such as a high power light source and a sensitive detector, such as a spectrophotometer. The remote sensor device may be useful in a localized area or over a large area of interest, in the atmosphere or in solution and may be deployed over the area of interest by any means known in the art. For example, the remote sensor device may be deployed over any area of interest by firing a rocket, or a mortar shell in the distance which releases the remote sensor device comprising a medium containing fragments of PCCA in water droplets wherein the remote sensor device is dispersed over the area of interest, e.g., dispersed into the environment over a battlefield or over a city or town.

The following examples are intended to illustrate the invention and should not be construed as limiting the invention in any way.

### EXAMPLES

### Example 1

PCCA Formation

Charged polystyrene particles were formed by placing approximately 60 g of polystyrene and about 2 g of 1-sodium, 1-allyloxy-2-hydroxypropane sulfonate into about 150 g of water. About 3 g of sodium-di(1,3-dimethylbutyl)sulfosuccinate, about 0.1 g of buffer and about 0.7 g of ammonium persulfate dissolved in about 5 ml of water were also added. The mixture was reacted for about 3 hours in a flask equipped with a stirring mechanism set at about 350 rpm at 70° C. The particles were about 105 nm in diameter and were purified by dialysis and ion exchange.

A portion of the colloid suspension was removed and further dialyzed for about one week in deionized water. The solution was then further purified by shaking with ion exchange resin until all of the impurity ions were removed and the CCA self assembled.

A PCCA was then made by adding to 3 ml of the CCA medium a mixture comprised of about 0.15 g AMD, about 0.07 g of 4-acrylamidobenzo 18-crown-6 ether, about 0.01 g of N,N′-methylenebisacrylamide and about 0.01 g of diethoxyacetophenone. The CCA/gel mixture was placed between two quartz plates. The plates were then exposed to UV light for about 5 minutes.

### Example 2

Lead Sensing

The response of the PCCA fragments described in Example 1 to very low concentrations of Pb(NO₃)₂ was determined. The sensor was dispersed into fragments of 25-250 μm using a tissue homogenizer (Biospec Products Inc. Model 985370 Bartlesville, Okla.). The homogenizer was operated for 1 to 5 minutes at a speed of 30,000 RPM. The longer the homogenizer was operated the smaller the fragments became. Likewise the fragments could be made by using a cell homogenizer as well as by using a mortar and pestle for a frozen sample. The sensor had a response, detectable by a spectrometer, of a 20 nm shift in response to a 250 μM concentration of Pb²⁺. The response of 45 nm to 500 μM Pb²⁺ is easily detectable with the unaided human eye. The PCCA is blue at lead concentrations of 200 μM and green at lead concentrations of 500 μM. The lead response reached a maximum at 7.5 mM. The shift in the PCCA diffraction over the entire detectable concentration range of lead is shown in FIGS. 6a and 6b. The fragments returned to their original diffraction color when washed by rinsing with deionized water. The water was removed by filtering the liquid through a 5.0 micron nylon syringe filter, which trapped the particles. Water was then flushed through this filter in the opposite direction to deposit the IPCCA gel fragments into a sample vial. This washing procedure was repeated 5 times between sample determinations.

### Example 3

pH Sensing

A sensing device was made by taking a blue diffracting suspension of polystyrene colloids prepared as described above in Example 1 and polymerizing said CCA of these colloids in an acrylamide gel. The PCCA was then immersed in a 0.1 M sodium hydroxide bath for about 10 minutes, which hydrolyzed some, but not all, of the CONH₂ groups to COOH. The hydrolyzed gel was washed in pure water to remove the sodium hydroxide. At this point, the hydrolyzed gel was swollen and diffracted in the red or infrared region. This gel was then fragmented into particles/fragments using a tissue homogenizer and tested as a pH detector. The fragments in dispersion in a medium diffracted at 500 nm at a pH of ˜2, and the diffraction wavelength increased to 600 nm at pH ˜7, further increased to 680 nm at pH 9.6, and then decreased to 650 nm at pH 10.6 and further decreased to 600 nm at pH of 11 illustrating a useful pH range of 2 to ˜10. See FIGS. 5a-5c.

### Example 4

Temperature Sensing

A sensing device was created according to Example 1 with NIPAM as monomer and with no molecular recognition component. Polymerization was done at 0° C. The sensor was dispersed into fragments using a tissue homogenizer and the fragments were used as a temperature sensor. At 7.2° C. the fragments diffract 625 nm light, while at 35.2° C. the fragments diffract 425 nm light. See FIGS. 4a and 4b. Between 7.2° C. to 25° C. the diffraction shifts from 625 nm to 560 nm, which gives a sensitivity of ˜3 nm/° C. A more sensitive response occurs around the phase transition temperature, with an 80 nm shift occurring over the 6 degree range of 27.5° C. to 33.5° C., giving a sensitivity of ˜17 nm/° C.

We claim:

1. A colorimetric reagent comprising:
a dispersion of fragments of a polymerized crystalline colloidal array in a medium wherein said polymerized crystalline colloidal array comprises a hydrogel that undergoes a volume change in response to a specific stimulus and a light diffracting crystalline colloidal array of charged particles polymerized in the hydrogel; the crystalline colloidal array having a lattice spacing that changes when the volume of said hydrogel changes, thereby causing the diffracted wavelength of the crystalline colloidal array to change.

2. The colorimetric reagent of claim 1, wherein said hydrogel comprises a first comonomer that is a gel monomer, a crosslinking agent and a molecular recognition component.

3. The colorimetric reagent of claim 2 wherein the molecular recognition component reacts with the stimulus to be detected.

4. The colorimetric reagent of claim 2, wherein said hydrogel is hydrophilic.

5. The colorimetric reagent of claim 2, wherein said gel monomer is ion-free.

6. The colorimetric reagent of claim 5, wherein said gel monomer is selected from the group consisting of acrylamide gels, purified agarose gels, N-vinylpyrolidone gels, and methacrylate gels.

7. The colorimetric reagent of claim 6, wherein said gel monomer is N-isopropylacrylamide.

8. The colorimetric reagent of claim 1, wherein said volume change is between about 0.1 and ˜300%.

9. The colorimetric reagent of claim 2, wherein said crosslinking agent is selected from the group consisting of N,N′-methylenebisacrylamide, methylenebismethacrylamide and ethyleneglycol-dimethacrylate.

10. The colorimetric reagent of claim 9, wherein said crosslinking agent is N,N′-methylenebisacrylamide.

11. The colorimetric reagent of claim 1, wherein said charged particles are selected from the group consisting of colloidal polystyrene, polymethylmethacrylate, silicon dioxide, aluminum oxide, polytetrafluoroethylene and poly N-isopropylacrylamide.

12. The colorimetric reagent of claim 1, wherein the stimulus is selected from the group consisting of lead ions and biological and chemical weapons.

13. The colorimetric reagent of claim 2, wherein said hydrogel further comprises a second monomer.

14. The colorimetric reagent of claim 13, wherein said second monomer is an acrylamide or a substituted acrylamide.

15. The colorimetric reagent of claim 2, further comprising one or more linking molecules that link the molecular recognition component to the gel monomer.

16. A method of making a colorimetric reagent comprising:
a) adding a gel monomer, a crosslinking agent and a polymerization initiator to a medium comprising a crystalline colloidal array formed by self assembly of charged colloidal particles to form a mixture;
b) polymerizing the mixture of step (a) to form a polymerized crystalline colloidal array wherein said polymerized crystalline colloidal array is embedded in a hydrogel;
c) fragmenting said polymerized crystalline colloidal array; and
d) adding a molecular recognition component to the product of step (c), wherein said hydrogel undergoes a volume change in response to a stimulus.

17. The method of claim 16, wherein said molecular recognition component is added to the product of step (b) by use of one or more linking molecules.

18. The method of claim 17, wherein said molecular recognition component is reacted with a linking molecule that can be bound to either a second linking molecule or to the gel.

19. The method of claim 16, further comprising hydrolyzing the polymerized crystalline colloidal array obtained in (b) before fragmenting the polymerized crystalline colloidal array.

20. The method of claim 16, further comprising a UV photoinitiator wherein the polymerization step is effected by exposing the mixture of step (a) to UV light from the UV photoinitiator.

21. The method of claim 16, further comprising a gel monomer selected from the group consisting of acrylamide gels, purified agarose gels, N-vinylpyrolidone gels, and methacrylate gels.

22. The method of claim 21, wherein the gel monomer is N-isopropylacrylamide.

23. The method of claim 16, further comprising a crosslinking agent selected from the group consisting of N,N′-methylenebisacrylamide, methylenebismethacrylamide and ethyleneglycol-dimethacrylate.

24. The method of claim 23 wherein said crosslinking agent is N,N′-methylenebisacrylamide.

25. The method of claim 16, further comprising charged particles selected from the group consisting of colloidal polystyrene, polymethylmethacrylate, silicon dioxide, aluminum oxide, polytetrafluoroethylene and poly N-isopropylacrylamide as said charged colloidal particles.

26. A remote sensor device comprising:
a dispersion of fragments of a polymerized crystalline colloidal array in a medium wherein said polymerized crystalline colloidal array comprises a hydrogel that undergoes a volume change in response to stimulus and a light diffracting crystalline colloidal array of charged particles polymerized in the hydrogel, the crystalline colloidal array having a lattice spacing that changes when the volume of the hydrogel changes, thereby causing the diffracted wavelength of the crystalline colloidal array to change.

27. The remote sensor device of claim 26 wherein the device detects temperature changes.

28. The remote sensor device of claim 26 wherein the stimulus is an analyte and wherein the device detects the presence of the analyte.

29. The remote sensor device of claim 26 further comprising a monitoring means.

30. The remote sensor device of claim 29 wherein the monitoring means comprises a high power light source and a sensitive detector.

31. The remote sensor device of claim 26 wherein said device is in the environment.

32. The remotes sensor of claim 26 wherein said hydrogel comprises a first comonomer that is a gel monomer, a crosslinking agent and a molecular recognition component.

33. The remote sensor device of claim 32 wherein the molecular recognition component reacts with the stimulus.

34. The remote sensor device of claim 33 wherein the stimulus is selected from the group consisting of chemical weapons and biological weapons.

35. The remote sensor device of claim 33 wherein the stimulus is an atmospheric contaminant.

36. A temperature sensing device comprising:
a dispersion of fragments of a polymerized crystalline colloidal array in a medium wherein the polymerized crystalline colloidal array comprises a hydrogel that undergoes a volume change in response to a change in temperature and a light diffracting crystalline colloidal array of charged particles polymerized in the hydrogel, the crystalline colloidal array having a lattice spacing that changes when said volume of said hydrogel changes, thereby causing the diffracted wavelength of the crystalline colloidal array to change.

37. The temperature sensing device of claim 36, wherein the hydrogel is comprised of a first comonomer that is a gel monomer, a crosslinking agent and a molecular recognition component.

38. The temperature sensing device of claim 37 wherein the molecular recognition component is acrylic acid.

49. The temperature sensing device of claim 38 wherein the acrylic acid can detect the change in temperature.

40. A gas sensing device comprising:
a dispersion of fragments of a polymerized crystalline colloidal array in a medium wherein the polymerized crystalline colloidal array comprises a hydrogel that undergoes a volume change in response to a gas and a light diffracting crystalline colloidal array of charged particles polymerized in the hydrogel, the crystalline colloidal array having a lattice spacing that changes when the volume of the hydrogel changes, thereby causing the diffracted wavelength of the crystalline colloidal array to change.

41. The gas sensing device of claim 40, wherein the hydrogel comprises a comonomer that is a gel monomer, a crosslinking agent, and a molecular recognition component.

42. The gas sensing device of claim 41 wherein the gel monomer is NIPAM.

43. The gas sensing device of claim 42 wherein the gas is water vapor.

44. The gas sensing device of claim 41 wherein the molecular recognition component is a gas binding component.

45. The gas sensing device of claim 44 wherein the gas binding component is glucose oxidase.

46. The gas sensing device of claim 45 wherein the gas is oxygen.

47. A pH sensing device comprising:
a dispersion of fragments of a polymerized crystalline colloidal array in a medium wherein the polymerized crystalline colloidal array comprises a hydrogel that undergoes a volume change in response to a change in pH and a light diffracting crystalline colloidal array of charged particles polymerized in the hydrogel, the crystalline colloidal array having a lattice spacing that changes when the volume of the hydrogel changes, thereby causing the diffracted wavelength of the crystalline colloidal array to change.

48. A lead sensing device comprising:
a dispersion of fragments of a polymerized crystalline colloidal array in a medium wherein the polymerized crystalline colloidal array comprises a hydrogel that undergoes a volume change in response to lead and a light diffracting crystalline colloidal array of charged particles polymerized in the hydrogel, the crystalline colloidal array having a lattice spacing that changes when the volume of said hydrogel changes, thereby causing the diffracted wavelength of the crystalline colloidal array to change.

49. A method for remote sensing of an environment comprising exposing a remote sensor device to the environment and monitoring the remote sensor device from a distance wherein the remote sensor device comprises:
a dispersion of fragments of a polymerized crystalline colloidal array in a medium wherein the polymerized crystalline colloidal array comprises a hydrogel that undergoes a volume change in response to a specific stimulus; and a light diffracting crystalline colloidal array of charged particles polymerized in the hydrogel, the crystalline colloidal array having a lattice spacing that changes when the volume of the hydrogel changes, thereby causing the diffracted wavelength of the crystalline colloidal array to change.

50. The method of claim 49 wherein the stimulus is a change in temperature and the remote sensor device detects the change in temperature.

51. The method of claim 49 wherein the stimulus is an analyte and the remote sensor device detects the presence of the analyte.

52. The method of claim 49 wherein the remote sensor device is monitored by a monitoring means.

53. The method of claim 52 wherein the monitoring means comprises a high power light source and a sensitive detector.

54. The method of claim 49 wherein the remote sensor device is deployed over an area of interest.

55. The method of claim 49 wherein the hydrogel is comprised of a first comonomer that is a gel monomer, a crosslinking agent and a second comonomer that is a molecular recognition component.

56. The method of claim 55 wherein the stimulus is a biological or chemical weapon.

57. The method of claim 56 wherein the molecular recognition component reacts with the biological and/or chemical weapon.

58. The method of claim 55 wherein the stimulus is an atmospheric contaminant.

59. The method of claim 58 wherein the molecular recognition component reacts with the atmospheric contaminant.

60. A method for detecting temperature changes comprising exposing a temperature sensing device to an environment wherein said temperature device comprises:
a dispersion of fragments of a polymerized crystalline colloidal array in a medium wherein said polymerized crystalline colloidal array comprises a hydrogel that undergoes a volume change in response to a temperature change and a light diffracting crystalline colloidal array of charged particles polymerized in the hydrogel, the crystalline colloidal array havin g a lattice spacing that changes when the volume of the hydrogel changes, thereby causing the diffracted wavelength of the crystalline colloidal array to change; and wherein said temperature sensor device can detect changes in temperature of the environment.

61. The method of claim 60, wherein the hydrogel comprises a first comonomer that is a gel monomer, a crosslinking agent and a second comonomer that is a molecular recognition component.

62. The method of claim 61 wherein the molecular recognition component is acrylic acid.

63. A method for detecting a gas in an environment comprising exposing a gas sensing device to the environment wherein the gas sensing device comprises:
a dispersion of fragments of a polymerized crystalline colloidal array in a medium wherein said polymerized crystalline colloidal array comprises a hydrogel that undergoes a volume change in response to the gas and a light diffracting crystalline colloidal array of charged particles polymerized in the hydrogel, the crystalline colloidal array having a lattice spacing that changes when the volume of said hydrogel changes, thereby causing the diffracted wavelength of the crystalline colloidal array to change; and wherein the gas sensing device can detect gas in the environment.

64. The method of claim 63, wherein the hydrogel comprises a comonomer that is a gel monomer and, crosslinking agent, and a molecular recognition component.

65. The method of claim 64 wherein the gel monomer is NIPAM.

66. The method of claim 65 wherein the gas is water vapor.

67. The method of claim 64 wherein the molecular recognition component is a gas binding component.

68. The method of claim 67 wherein the gas binding component is glucose oxidase.

69. The method of claim 68 wherein the gas is oxygen.

70. A method for detecting the pH of an environment comprising exposing a pH sensing device to the environment wherein the pH sensing device comprises:
a dispersion of fragments of a polymerized crystalline colloidal array in a medium wherein said polymerized crystalline colloidal array comprises a hydrogel that undergoes a volume change in response to a change in pH and a light diffracting crystalline colloidal array of charged particles polymerized in the hydrogel, the crystalline colloidal array having a lattice spacing that changes when the volume of the hydrogel changes, thereby causing the diffracted wavelength of the crystalline colloidal array to change; and wherein said pH sensing device detects the pH of the environment.

71. The method of claim 70 wherein the environment is a solution.

72. The method of claim 70 further comprising an ionic concentration sensing device wherein said ionic concentration sensing device can detect the ionic concentration of the environment.

73. The method of claim 72 wherein the pH sensing device is calibrated according to the ionic concentration of the environment.

74. A method for detecting lead in an environment comprising exposing a lead sensing device to the environment wherein the lead sensing device comprises:
a dispersion of fragments of a polymerized crystalline colloidal array in a medium wherein said polymerized crystalline colloidal array comprises a hydrogel that undergoes a volume change in response to lead and a light diffracting crystalline colloidal array of charged particles polymerized in the hydrogel, the crystalline colloidal array having a lattice spacing that changes when the volume of the hydrogel changes, thereby causing the diffracted wavelength of the crystalline colloidal array to change; and wherein said lead sensing device can detect the presence of lead in the environment.
